SEQUENCE LISTING (15 sequences)

SEQ ID NO: 1 - DNA, 99 bp, Homo sapiens
  human urodilatin, atrial natriuretic peptide (ANP)95-126 fragment (ANP(95-126))
actgcccctcggagcctgcggagatccagctgcttcgggggcaggatggacaggattggagcccagagcggactgggctgtaacagcttccggtactga

SEQ ID NO: 2 - protein, 32 aa, Homo sapiens
  human urodilatin, atrial natriuretic peptide (ANP)95-126 fragment (ANP(95-126))
TAPRSLRRSSCFGGRMDRIGAQSGLGCNSFRY

SEQ ID NO: 3 - DNA, 681 bp, Homo sapiens
  human Fc IgG1(m1), human IgG1 heavy chain constant region, human immunoglobulin heavy chain constant gamma 1 (G1m marker) (HuFcG1m(1))
gacaaaactcacacatgcccaccgtgcccagcacctgaagccgagggcgcgccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggcaggagttgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 4 - protein, 227 aa, Homo sapiens
  human Fc IgG1(m1), human IgG1 heavy chain constant region, human immunoglobulin heavy chain constant gamma 1 (G1m marker) (HuFcG1m(1))
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRQELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 780 bp, Artificial Sequence
  Description of Artificial SequenceFc- urodilatin fusion protein, Fc-Uro huFcG1(m1) fusion (without signal peptide), Fc portion of Ig heavy chain constant gamma 1 region (G1m marker) N terminus and urodilatin C terminus
gacaaaactcacacatgcccaccgtgcccagcacctgaagccgagggcgcgccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggcaggagttgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaactgcccctcggagcctgcggagatccagctgcttcgggggcaggatggacaggattggagcccagagcggactgggctgtaacagcttccggtactga

SEQ ID NO: 6 - protein, 259 aa, Artificial Sequence
  Description of Artificial SequenceFc- urodilatin fusion protein, Fc-Uro huFcG1(m1) fusion (without signal peptide), Fc portion of Ig heavy chain constant gamma 1 region (G1m marker)N terminus and urodilatin C terminus
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRQELTKNQVSLTCLVKGFYPSDIAVEWESNGQPENNYKTTPPVLDSDGSFFLYSKLTVDKSRWQQGNVFSCSVMHEALHNHYTQKSLSLSPGKTAPRSLRRSSCFGGRMDRIGAQSGLGCNSFRY

SEQ ID NO: 7 - DNA, 855 bp, Artificial Sequence
  Description of Artificial SequenceFc- urodilatin fusion protein, Fc-Uro huFcG1(m1) fusion (including signal peptide),Fc portion of Ig heavy chain constant gamma 1 region (G1m marker) N terminus and pro-ANP signal and urodilatin C terminus
atgagctccttctccaccaccaccgtgagcttcctccttttactggcattccagctcctaggtcagaccagagctgacaaaactcacacatgcccaccgtgcccagcacctgaagccgagggcgcgccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggcaggagttgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaaactgcccctcggagcctgcggagatccagctgcttcgggggcaggatggacaggattggagcccagagcggactgggctgtaacagcttccggtactga

SEQ ID NO: 8 - protein, 284 aa, Artificial Sequence [sequence omitted]

SEQ ID NO: 9 - DNA, 9 bp, Artificial Sequence
  Description of Artificial Sequencelinker sequence
gcggccgcg

SEQ ID NO: 10 - protein, 3 aa, Artificial Sequence
  Description of Artificial Sequencelinker sequence
AAA

SEQ ID NO: 11 - DNA, 789 bp, Artificial Sequence
  Description of Artificial Sequenceurodilatin- Fc fusion protein, Uro-Fc huFcG1(m1) fusion (without signal peptide),urodilatin N terminus and Fc portion of Ig heavy chain constant gamma 1 region (G1m marker) C terminus
actgcccctcggagcctgcggagatccagctgcttcgggggcaggatggacaggattggagcccagagcggactgggctgtaacagcttccggtacgcggccgcggacaaaactcacacatgcccaccgtgcccagcacctgaagccgagggcgcgccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggcaggagttgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 12 - protein, 262 aa, Artificial Sequence
  Description of Artificial Sequenceurodilatin- Fc fusion protein, Uro-Fc huFcG1(m1) fusion (without signal peptide), urodilatin N terminus and Fc portion of Ig heavy chain constant gamma 1 region (G1m marker) C terminus
TAPRSLRRSSCFGGRMDRIGAQSGLGCNSFRYAAADKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRQELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 864 bp, Artificial Sequence
  Description of Artificial Sequenceurodilatin- Fc fusion protein, Uro-Fc huFcG1(m1) fusion (including signal peptide),pro-ANP signal and urodilatin N terminus and Fc portion of Ig heavy chain constant gamma 1 region (G1m marker) C terminus
atgagctccttctccaccaccaccgtgagcttcctccttttactggcattccagctcctaggtcagaccagagctactgcccctcggagcctgcggagatccagctgcttcgggggcaggatggacaggattggagcccagagcggactgggctgtaacagcttccggtacgcggccgcggacaaaactcacacatgcccaccgtgcccagcacctgaagccgagggcgcgccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccggcaggagttgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 14 - protein, 287 aa, Artificial Sequence
  Description of Artificial Sequenceurodilatin- Fc fusion protein, Uro-Fc huFcG1(m1) fusion (including signal peptide),pro-ANP signal and urodilatin N terminus and Fc portion of Ig heavy chain constant gamma 1 region (G1m marker) C terminus
MSSFSTTTVSFLLLLAFQLLGQTRATAPRSLRRSSCFGGRMDRIGAQSGLGCNSFRYAAADKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRQELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 200 aa, Artificial Sequence
  Description of Artificial Sequencepoly-Gly flexible linker, tag
GGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGGG

(19) United States
(12) Patent Application Publication
(10) Pub. No.: US 2008/0181903 A1
(43) Pub. Date: Jul. 31, 2008

(54) CONJUGATE OF NATRIURETIC PEPTIDE AND ANTIBODY CONSTANT REGION

(75) Inventors: Vinay Bhaskar, San Francisco, CA (US); Robert Bryan Dubridge, Belmont, CA (US); Vanitha Ramakrishnan, Belmont, CA (US); Hans-Michael Jantzen, San Francisco, CA (US)

Correspondence Address: TOWNSEND AND TOWNSEND AND CREW LLP, TWO EMBARCADERO CENTER, EIGHTH FLOOR, SAN FRANCISCO, CA, 94111-3834, US

(73) Assignee: PDL BioPharma, Inc., Fremont, CA (US)

(21) Appl. No.: 11/960,427

(22) Filed: Dec. 19, 2007

Related U.S. Application Data
(60) Provisional application No. 60/876,913, filed on Dec. 21, 2006.

(51) Int. Cl. A61K 39/395; C07K 16/18; C12N 15/11; C12N 15/00; A61P 9/00; A61P 1/00; C12N 5/06; C12P 21/04

(52) U.S. Cl. 424/178.1; 530/391.1; 536/23.53; 435/320.1; 435/325; 435/69.7

(57) ABSTRACT

The present application describes a conjugate between a natriuretic peptide, such as urodilatin, and the constant region of an immunoglobulin or a fragment thereof. Also described are compositions comprising the conjugate and methods for using the conjugate.

This application claims priority to U.S. Provisional Patent Application No. 60/876,913, filed Dec. 21, 2006, the contents of which are hereby incorporated by reference in the entirety.

### BACKGROUND OF THE INVENTION

A family of related peptides has been discovered that works in concert to achieve salt and water homeostasis in the body. These peptides, termed natriuretic peptides for their role in moderating natriuresis and diuresis, have varying amino acid sequences and originate from different tissues within the body. This family of natriuretic peptides includes atrial natriuretic peptide (ANP), brain natriuretic peptide (BNP), C-type natriuretic peptide (CNP), Dendroaspis natriuretic peptide (DNP), and urodilatin (URO, or ularitide). Their tissue-specific distribution is as follows: heart (ANP, BNP, and DNP); brain (ANP, BNP, and CNP); endothelial cells (CNP); plasma (DNP); and kidney (URO). These peptides are constituents of a hormonal system that plays a critical role in maintaining an intricate balance of blood volume/pressure in the human body.

Urodilatin, like ANP, is derived from the precursor protein pro-ANP, and is secreted by kidney cells. Urodilatin promotes excretion of sodium and water by acting directly on kidney cells in the collecting duct to inhibit sodium and water reabsorption. Like other natriuretic peptides, such as ANP and BNP, urodilatin has been studied for use in treating various conditions, including bacterial infections, pulmonary and bronchial diseases, renal failure, and congestive heart failure (see, e.g., U.S. Pat. Nos. 5,571,789 and 6,831,064; U.S. patent application published as US2005/0089514; PCT application published as WO2006/110743; Kentsch et al., Eur. J. Clin. Invest. 1992, 22(10):662-669; Kentsch et al., Eur. J. Clin. Invest. 1995, 25(4):281-283; Elsner et al., Am. Heart J 1995, 129(4):766-773; and Forssmann et al., Clinical Pharmacology and Therapeutics 1998, 64(3):322-330).

Given the various therapeutic applications of natriuretic peptides, there exists the need for developing modified versions of natriuretic peptides or their derivatives that retain the biological activity of the peptide, and have other advantageous properties, such as a longer serum half-life. The present invention addresses this and other related needs by providing a novel conjugate of natriuretic peptide-antibody constant region.

### BRIEF SUMMARY OF THE INVENTION

In one aspect, the present invention relates to a novel conjugate comprising a natriuretic peptide and an antibody constant region. The conjugate binds to an NPR (e.g., NPR-A) receptor. In some cases, the natriuretic peptide and the antibody constant region are linked via a linker. In other cases, the conjugate is a direct fusion polypeptide, which may be the result of a direct fusion between the natriuretic peptide and the antibody constant region, or may contain a peptide linker. In some exemplary embodiments, the natriuretic peptide is urodilatin. In other exemplary embodiments, the conjugate is a fusion polypeptide, which comprises the amino acid sequence of SEQ ID NO:6 or 12. In this conjugate, the antibody constant region may be linked to the N-terminus or C-terminus of the natriuretic peptide, or the natriuretic peptide may be linked to an internal amino acid of the antibody constant region. Moreover, the conjugate may contain more than one antibody constant region. For instance, two antibody constant regions, which may be identical or different from each other, can be present in a conjugate with a natriuretic peptide. The constant regions may be located at both its N-terminus and C-terminus to sandwich the natriuretic peptide, or may be located at the same (N-terminus or C-terminus) of the natriuretic peptide.

In addition, the conjugate can be a monomer or a dimer, and the antibody constant region can be derived from either an antibody heavy chain or light chain. In one exemplary embodiment, the conjugate is a homodimer consisting of two identical strains of fusion polypeptide of natriuretic peptide-antibody heavy chain constant region, connected via a disulfide bond located within the heavy chain constant region. In another embodiment, the conjugate is a heterodimer of two distinct strains of fusion polypeptide: one of natriuretic peptide-antibody heavy chain constant region fusion and the other of natriuretic peptide-antibody light chain constant region fusion, connected via a disulfide bond located within the heavy and light chain constant regions.

Beyond the ability to bind to an NPR-A receptor, the conjugate preferably is also capable of activating the NPR-A receptor, leading to an increase in the intracellular cGMP level. In comparison with an unmodified natriuretic peptide, the conjugate demonstrates an increased serum half-life.

In another aspect, the present invention relates to an isolated nucleic acid comprising a polynucleotide sequence encoding a fusion polypeptide that comprises a natriuretic peptide and an antibody constant region. In an exemplary embodiment, the natriuretic peptide is urodilatin. In another exemplary embodiment, the nucleic acid comprises the polynucleotide sequence of SEQ ID NO:5 or 11.

The invention also relates to an expression cassette comprising the nucleic acid described above, an isolated host cell transfected with the expression cassette. The host cell may be a prokaryotic cell or a eukaryotic cell.

In an additional aspect, the present invention relates to a method for recombinantly producing a fusion polypeptide comprising a natriuretic peptide and an antibody constant region. The method comprises the following steps: a. transfecting a host cell with an expression cassette described above; and b. culturing the cell under the condition that are suitable for the cell to express the fusion polypeptide. In one example, the natriuretic peptide is urodilatin.

In a further aspect, the present invention relates to a composition comprising (1) a conjugate of a natriuretic peptide and an antibody constant region and (2) a pharmaceutically acceptable carrier. The invention also relates to a method for treating bacterial infection, pulmonary and bronchial diseases, renal failure, or heart failure, comprising the step of administering to a patient in need thereof an effective amount of a conjugate a natriuretic peptide and an antibody constant region. In some embodiments, the conjugate is a fusion protein.

### BRIEF DESCRIPTION OF THE DRAWINGS

FIG. 1A DNA and protein sequences of Uro-Fc(huFcG1(m1)). 1B DNA and protein sequences of Fc-Uro(huFcG1(m1)).

FIG. 2A Binding of Uro-Fc (huFcG1(m1)) and ularitide to purified NPR-A-Fc fusion proteins. 2B Binding of Fc-Uro(huFcG1(m1)) and ularitide to purified NPR-A-Fc fusion proteins.

FIG. 3A Uro-Fc (huFcG1(m1)) and ularitide activate NPR-A receptors expressed on transfected cells. 3B Fc-Uro(huFcG1(m1)) and ularitide activate NPR-A receptors expressed on transfected cells.

### DEFINITIONS

An “antibody” refers to a glycoprotein of the immunoglobulin family or a polypeptide comprising fragments of an immunoglobulin that is capable of noncovalently, reversibly, and in a specific manner binding a corresponding antigen. An exemplary antibody structural unit comprises a tetramer. Each tetramer is composed of two identical pairs of polypeptide chains, each pair having one “light” (about 25 kD) and one “heavy” chain (about 50-70 kD), connected through a disulfide bond. The recognized immunoglobulin genes include the κ, λ, α, γ, δ, ε, and μ constant region genes, as well as the myriad immunoglobulin variable region genes. Light chains are classified as either κ or λ Heavy chains are classified as γ, μ, α, δ, or ε, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (V_{L}) and variable heavy chain (V_{H}) refer to these regions of light and heavy chains, respectively, whereas C_{L} and C_{H} refer to the constant regions of the light chain and heavy chain, respectively. There is one constant domain within each light chain constant region or C_{L}, whereas there are 3 or 4 constant domains (C_{H}1, C_{H}2, C_{H}3, and C_{H}4) within each heavy chain constant region or C_{H}, depending on the type of heavy chain (γ, μ, α, δ, or ε).

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified. In particular, an isolated gene is separated from open reading frames that flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

A “natriuretic peptide” is a peptide that has the biological activity of promoting natriuresis, diuresis, and vasodilation. Assays for testing such activity are known in the art, e.g., as described in U.S. Pat. Nos. 4,751,284 and 5,449,751. Examples of natriuretic peptides include, but are not limited to, atrial natriuretic peptide (ANP(99-126)), brain natriuretic peptide (BNP), C-type natriuretic peptide (CNP), Dendroaspis natriuretic peptide (DNP), urodilatin (URO, or ularitide), and any fragments of the prohormone ANP(1-126) or BNP precursor polypeptide that retains the vasodilating, natriuretic, or diuretic activity. For further description of exemplary natriuretic peptides and their use or preparation, see, e.g., U.S. Pat. Nos. 4,751,284, 4,782,044, 4,895,932, 5,449,751, 5,461,142, 5,571,789, and 5,767,239. See also, Ha et al., Regul. Pept. 133(1-3):13-19, 2006. As used in this application, the term “natriuretic peptide” also broadly encompasses a peptide having an amino acid sequence substantially identical (for instance, having a sequence identity at least 80% or 85%, more preferably at least 90%, 95%, or even higher) to a naturally occurring natriuretic peptide (e.g., ANP or URO). Typically, such a peptide may include one, two, three, four, or up to five amino acids that have been modified from the naturally occurring sequence by addition, deletion, or substitution. Furthermore, the term “natriuretic peptide” encompasses any peptide having the amino acid sequence of a naturally occurring natriuretic peptide with chemical modification (e.g., deamination, phosphorylation, PEGylation, etc.) at one or more residues or substitution by the corresponding D-isomer(s), so long as the peptide retains a substantial portion, e.g., at least 1%, preferably 10%, more preferably 50%, and most preferably at least 80%, 90% or higher, of the biological activity of the corresponding wild-type natriuretic peptide.

The term “urodilatin” generally refers to a 32-amino acid peptide hormone that is described by U.S. Pat. No. 5,449,751 and has the amino acid sequence set forth in GenBank Accession No. 1506430A. Urodilatin, the 95-126 fragment of atrial natriuretic peptide (ANP), is also referred to as ANP(95-126). The term “atrial natriuretic peptide” or “ANP(99-126)” refers to a 28-amino acid peptide hormone, which is transcribed from the same gene and derived from the same polypeptide precursor, ANP(1-126), as urodilatin but without the first four amino acids at the N-terminus. For a detailed description of the prohormone, see, e.g., Oikawa et al. (Nature 1984; 309:724-726), Nakayama et al. (Nature 1984; 310:699-701), Greenberg et al. (Nature 1984; 312:656-658), Seidman et al. (Hypertension 1985; 7:31-34) and GenBank Accession Nos. 1007205A, 1009248A, 1101403A, and AAA35529. The polynucleotide sequence encoding this prohormone is provided in GenBank Accession No. NM_{—}6172.1. Conventionally, the term urodilatin (URO) is more often used to refer to the naturally occurring peptide, whereas the term “ularitide” is often used to refer to the recombinantly produced or chemically synthesized peptide. In this application, the term “urodilatin” and “ularitide” are used interchangeably to broadly encompass both a naturally occurring peptide and a recombinant or synthetic peptide. The terms also encompass any peptide of the above-cited amino acid sequence containing chemical modification (e.g., deamination, phosphorylation, PEGylation, etc.) at one or more residues or substitution by the corresponding D-isomer(s), so long as the peptide retains the biological activity as a natriuretic peptide. Furthermore, a chemically modified urodilatin or ularitide may contain one or two amino acid substitutions for the purpose of facilitating the desired chemical modification (e.g., to provide a reactive group for conjugation). “Urodilatin” or “ularitide” of this application, regardless of whether it contains chemical modifications or amino acid sequence modification, retains a substantial portion, i.e., at least 1%, preferably 10%, more preferably 50%, and most preferably at least 80% or 90%, of the biological activity of the naturally-occurring wild-type urodilatin or ANP(95-126).

As used herein, an “antibody (or immunoglobulin) constant region” refers to a polypeptide that corresponds to at least a portion of the constant region of an antibody heavy chain or light chain, such portion including at least one constant domain (e.g., the constant domain of C_{L} or one of the constant domains of C_{H}). For example, an “antibody constant region” used for making the conjugates of this invention may be derived from an antibody heavy chain and include two out of three (C_{H}2 and C_{H}3 for IgA, IgD, and IgG) or three out of four (C_{H}2, C_{H}3, and C_{H}4, for IgE and IgM) constant domains; the first constant domain (C_{H}1) may be present in some cases but may be excluded in others. Such an antibody constant region can be obtained by a variety of means, e.g., by a recombinant method or synthetic method, or by purification subsequent to enzymatic digestion, for instance, pepsin or papain digestion of an intact antibody or an antibody heavy or light chain. Further encompassed by this term as used in this application are polypeptides having a substantial sequence identity (for instance, at least 80%, 85%, 90%, 95% or more) to the corresponding amino acid sequence of an antibody heavy or light chain constant region or a portion thereof that contains at least one constant domain nearest to the C-terminus of the antibody chain, so long as the presence of such an “antibody constant region” in a natriuretic peptide-antibody constant region conjugate renders the conjugate a higher serum stability, e.g., at least 20%, preferably at least 30%, 50%, 80%, 100%, 200% or more, increase in the serum half-life when compared with the natriuretic peptide without the antibody constant region under the same conditions. A human antibody, e.g., a human IgG, is frequently used to derive a constant region or a fragment thereof for the purpose of making a natriuretic peptide conjugate of this invention.

A “conjugate,” as used in this application, refers to a compound having at least one natriuretic peptide and one antibody constant region joined at the polypeptide level, with or without the use of a linker. A conjugate may be a fusion polypeptide produced as the result of joining at the nucleic acid level of genes encoding at least one natriuretic peptide and one antibody constant region, with or without a coding sequence for a peptide linker.

An “NPR-A receptor,” as used in this application, refers to natriuretic peptide receptor type A. Also termed guanylyl cyclase A, it is a transmembrane protein that is expressed on cell surface and synthesizes cGMP in response to hormone stimulation. See, e.g., Chinkers et al., Nature 338:78-83, 1989 and Lowe et al., EMBO J. 8(5):1377-84, 1989. NPR-B (guanylyl cyclase B) and NPR-C (a non-guanylyl cyclase) are two other members of the NPR family.

A “natriuretic peptide receptor” or an “NPR” is a receptor family that includes three receptors: NPR-A (guanylyl cyclase A), NPR-B (guanylyl cyclase B) and NPR-C (a non-guanylyl cyclase).

In this application, a conjugate comprising a natriuretic peptide (e.g., urodilatin) and an antibody constant region is said to “bind” an NPR-A receptor when the conjugate demonstrates a binding affinity to the NPR-A receptor similar to that of the corresponding wild-type natriuretic peptide (e.g., urodilatin) under the same assay conditions. For instance, a urodilatin-antibody constant region conjugate of this invention has at least 0.1%, 1%, 10%, or 20%, preferably at least 25% or 30%, and more preferably at least 50% or even higher, of the binding affinity exhibited by the wild-type urodilatin in an NPR-A binding assay.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994). The term nucleic acid is used interchangeably with gene, cDNA, and mRNA encoded by a gene.

The term “gene” means the segment of DNA involved in producing a polypeptide chain; it includes regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that function in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-TUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins, W.H. Freeman and Co., N.Y. (1984)).

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left most residue, which is numbered 1, in an unmodified wild-type polypeptide sequence.

As used in herein, the terms “identical” or percent “identity,” in the context of describing two or more polynucleotide or amino acid sequences, refer to two or more sequences or subsequences that are the same or have a specified percentage of amino acid residues or nucleotides that are the same (for example, a ularitide peptide used for making the conjugate of the present invention has an amino acid sequence substantially identical to the sequence of the naturally occurring urodilatin (SEQ ID NO:2), having at least 80% or 85%, preferably at 90% identity, more preferably at least 91%, 92%, 93%, 94%, 95%, 96%, 97%, 98%, 99%, or 100% identity, to SEQ ID NO:2; whereas the constant region polypeptide in the conjugate has an amino acid sequence substantially identical to that of the full length or a portion of a naturally occurring antibody heavy or light chain constant region, for instance, SEQ ID NO:4, having at least 80% or 85%, preferably at 90% identity, more preferably at least 91%, 92%, 93%, 94%, 95%, 96%, 97%, 98%, 99%, or 100% identity, to SEQ ID NO:4), when compared and aligned for maximum correspondence over a comparison window, or designated region as measured using one of the following sequence comparison algorithms or by manual alignment and visual inspection. As an example, an amino acid sequence substantially identical to SEQ ID NO:2 or 4 may be a sequence derived from SEQ ID NO:2 or 4 by substituting, deleting, or adding 1, 2, 3, 4, or up to 5 amino acids in SEQ ID NO:2, or by or substituting, deleting, or adding 1, 2, 3, 4, 5, 6, 7, 8, 9, or up to 10 amino acids in SEQ ID NO:4. The sequences substantially identical to the amino acid sequence of a naturally occurring natriuretic peptide or an antibody constant region, such as SEQ ID NO:2 or 4, are suitable for use in making the conjugates of this invention. With regard to polynucleotide sequences, the definition of identity also refers to the complement of a test sequence. Preferably, the identity exists over a region that is at least about 50 amino acids or nucleotides in length, or more preferably over a region that is 75-100 or 100-150 (e.g., about 120) amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of a natriuretic peptide or an antibody constant region used in conjugation with an exemplary amino acid sequence of, e.g., SEQ ID NO:2 or 4, respectively, the BLAST and BLAST 2.0 algorithms and the default parameters discussed below are used.

A “comparison window”, as used herein, includes reference to a segment of any one of the number of contiguous positions selected from the group consisting of 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the website of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff& Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

“Polypeptide” and “peptide” are used interchangeably herein to refer to a polymer of amino acid residues; whereas “protein” may contain one or multiple polypeptide chains. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

An “expression cassette” is a nucleic acid construct, generated recombinantly or synthetically, with a series of specified nucleic acid elements that permit transcription of a particular polynucleotide sequence (e.g., one encoding for a natriuretic peptide-antibody constant region fusion protein of this invention) in a host cell. An expression cassette may be part of a plasmid, viral genome, or nucleic acid fragment. Typically, an expression cassette includes a polynucleotide to be transcribed, operably linked to a promoter, and, optionally, other transcription regulatory elements such as an enhancer.

The term “administration” or “administering” refers to various methods of contacting a substance with an animal, such as a mammal, especially a human. Modes of administration may include, but are not limited to, methods that involve contacting the substance intravenously, intraperitoneally, intranasally, transdermally, topically, subcutaneously, parentally, intramuscularly, orally, or systemically, and via injection, ingestion, inhalation, implantation, or adsorption by any other means. The candidate therapeutic agent can be formulated as a pharmaceutical composition in the form of a syrup, an elixir, a suspension, a powder, a granule, a tablet, a capsule, a lozenge, a troche, an aqueous solution, a cream, an ointment, a lotion, a gel, or an emulsion. One exemplary means of administration of a natriuretic peptide conjugate of this invention is via intravenous delivery, where the conjugate can be formulated as a pharmaceutical composition in the form suitable for intravenous injection, such as an aqueous solution, a suspension, or an emulsion, etc. Other means for delivering a natriuretic peptide conjugate of this invention includes intradermal injection, subcutaneous injection, intramuscular injection, or transdermal or transmucosal application as in the form of a cream, a patch, or a suppository.

An “effective amount” refers to the amount of an active ingredient, e.g., a urodilatin-Fc fusion protein of this invention, in a pharmaceutical or physiological composition that is sufficient to produce a beneficial or desired effect at a level that is readily detectable by a method commonly used for detection of such an effect. Preferably, such an effect results in a change of at least 10% from the value of a basal level where the active ingredient is not administered, more preferably the change is at least 20%, 50%, 80%, or an even higher percentage from the basal level. As will be described below, the effective amount of an active ingredient may vary from subject to subject, depending on age, general condition of the subject, the severity of the condition being treated, and the particular biologically active agent administered, and the like. An appropriate “effective” amount in any individual case may be determined by one of ordinary skill in the art by reference to the pertinent texts and literature and/or by using routine experimentation.

A “pharmaceutically acceptable carrier” or “excipient” is an inert ingredient used in the formulation of a composition of this invention, which contains the active ingredient(s), e.g., a natriuretic peptide fusion protein of this invention. Such a carrier or excipient may act to stabilize the active ingredient and may be suitable for use, e.g., by injection into a patient in need thereof. This inert ingredient may be a substance that, when included in a composition of this invention, provides a desired pH, consistency, color, smell, or flavor of the composition. A pharmaceutically acceptable carrier can include, but is not limited to, carbohydrates (such as glucose, sucrose, or dextrans), antioxidants (such as ascorbic acid or glutathione), chelating agents, low molecular weight proteins, high molecular weight polymers, gel-forming agents, or other stabilizers and additives. Other examples of a pharmaceutically acceptable carrier include wetting agents, emulsifying agents, dispersing agents, or preservatives, which are particularly useful for preventing the growth or action of microorganisms. Various preservatives are well known and include, for example, phenol and ascorbic acid. Examples of carriers, stabilizers, or adjuvants can be found in Remington's Pharmaceutical Sciences, Mack Publishing Company, Philadelphia, Pa., 17th ed. (1985).

As used herein, a “patient” refers to a human or a non-human mammal.

An “increase in serum half-life” is a positive change in circulating half-life of a modified biologically active molecule (e.g., the conjugate of this invention, comprising a natriuretic peptide and an antibody constant region) relative to its non-modified form (e.g., the natriuretic peptide alone). Serum half-life is measured by taking blood samples at various time points after administration of the biologically active molecule, and determining the concentration of that molecule in each sample. Correlation of the serum concentration with time allows calculation of the serum half-life. The increase is desirably at least about 10% to 20%, or at least about 50% to 100%. Preferably the increase is at least about 3-fold, more preferably at least about 5-fold, and most preferably at least about 10-fold or higher.

### DETAILED DESCRIPTION OF THE INVENTION

### I. Introduction

The present inventors successfully produced conjugates between a natriuretic peptide, such as urodilatin (GenBank Accession No. 1506430), and a polypeptide corresponding to at least a portion of an immunoglobulin heavy or light chain constant region, and demonstrated that the conjugates retain the biological activity of the natriuretic peptide, for instance, the activity of urodilatin, in terms of its specific binding specificity for its natural receptor, NPR-A receptor, and its ability to induce increased intracellular cGMP level upon binding to the receptor. Because the antibody constant region portion of the conjugate prolongs the half-life of the conjugate, making the conjugate more stable in a patient's circulation than an unconjugated natriuretic peptide alone, the conjugate comprising the antibody constant region provides a more effective alternative to the unconjugated natriuretic peptide in the treatment of conditions for which the use of the natriuretic peptide is indicated.

There are several methods for producing a conjugate of the present invention, which comprises a natriuretic peptide and an antibody constant region. For example, one skilled in the art will recognize that when genes encoding the natriuretic peptide and the constant region are joined at nucleic acid level as a combined coding sequence for a fusion protein, a natriuretic peptide-antibody constant region fusion protein can be expressed in transfected cells. On the other hand, the natriuretic peptide portion and the antibody constant region may be joined at polypeptide level, after their separate production, by a variety of means such as recombinant production, chemical synthesis, or purification from a natural source following enzymatic digestion(s) as needed. The separately produced natriuretic peptide and antibody constant region may be joined by a direct chemical bond (peptide bond or non-peptide bond, such as a disulfide bond), or via one or more chemical linkers. Either the N- or C-terminal amino acid of the peptides can provide the linkage site, or an amino acid located in the middle of either peptide sequence, i.e., an internal amino acid, can provide the linkage site. As a third option, a natriuretic peptide-antibody constant region fusion protein may be directly synthesized as a single peptide by a chemical method known in the art.

### II. Recombinant Production of Natriuretic Peptide, Antibody Constant Region, or Their Fusion Protein

### A. General Recombinant Technology

The present invention utilizes numerous routine technologies in molecular and cellular biology. Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a polynucleotide encoding a natriuretic peptide, an antibody constant region, or their fusion protein and synthetic oligonucleotides can be verified using, e.g., the chain termination method for sequencing double-stranded templates of Wallace et al., Gene 16: 21-26 (1981).

### B. Generating Coding Sequence for a Natriuretic Peptide or Antibody Constant Region

Since the amino acid sequences of naturally occurring natriuretic peptides are known in the art, their encoding polynucleotide sequences are also known or can be easily derived. For instance, the polynucleotide sequence encoding urodilatin is well known in the art and provided in this application as SEQ ID NO:1, with the corresponding amino acid sequence set forth in SEQ ID NO:2. A number of sequences encoding the constant regions and individual constant domains of the heavy or light chains of IgA, IgD, IgE, IgG, and IgM antibodies of human or other species have also been identified. One exemplary sequence encoding a portion of a human IgG1 heavy chain constant region is provided in this application as SEQ ID NO:3, with the corresponding amino acid sequence set forth in SEQ ID NO:4.

Utilizing well known methods in the art, polynucleotide sequences encoding variants of these sequences can be readily generated. For instance, a PCR-based mutagenesis method permits one to produce coding sequences for variants with a substantial identity (e.g., at least 80%, 85%, 90% or 95% sequence identity) to the amino acid sequence of SEQ ID NO:2. Similarly, polynucleotide sequences encoding variants of an antibody constant region can be generated, which may have a sequence identity to these exemplary sequences ranging from 90%, 91%, 92%, 93%, 94%, 95%, 96%, 97%, 98%, to 99% or even higher.

Upon acquiring a polynucleotide sequence encoding a natriuretic peptide, an antibody constant region, or a fusion protein thereof as described in the present application, a skilled artisan can then subclone the polynucleotide sequence into a vector, for instance, an expression vector, so that a recombinant polypeptide can be produced from the resulting construct. Further modifications to the coding sequence, e.g., nucleotide substitutions, may be subsequently made to alter the characteristics of the recombinant polypeptide.

### C. Modification of Nucleic Acids for Preferred Codon Usage in a Host Organism

The polynucleotide sequence encoding a natriuretic peptide, an antibody constant region, or a fusion protein thereof can be further altered to coincide with the preferred codon usage of a particular host. For example, the preferred codon usage of one strain of bacterial cells can be used to derive a polynucleotide that encodes a natriuretic peptide-antibody constant region fusion protein of the invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute, Japan). This analysis is preferably limited to genes that are highly expressed by the host cell.

At the completion of modification, the coding sequences are verified by sequencing and are then subcloned into an appropriate expression vector for recombinant production of a desired polypeptide, e.g., a fusion protein comprising urodilatin and a Fc fragment.

### III. Expression and Purification of a Recombinant Protein

Following verification of the coding sequence, the desired recombinant protein, e.g., a natriuretic peptide-antibody constant region fusion protein of the present invention, can be produced using routine techniques in the field of recombinant genetics.

### A. Cells for Expression of the Recombinant Polypeptide

Various cell types, both prokaryotic and eukaryotic, are suitable for the expression of the recombinant protein of the present invention. These cell types include but are not limited to, for example, a variety of bacteria such as E. coli, Bacillus sp., and Salmonella, as well as eukaryotic cells such as yeast, insect cells, and mammalian cells (e.g., CHO cells). In some cases, plant cells are also appropriate as host cells for recombinant expression of a recombinant polypeptide. Suitable cells for gene expression are well known to those of skill in the art and are described in numerous scientific publications such as Sambrook and Russell, supra.

### B. Expression Systems

To obtain high level expression of a nucleic acid encoding a recombinant polypeptide of the present invention, one typically subclones a polynucleotide encoding the polypeptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function.

In addition to the promoter, the expression vector typically includes a transcription unit or expression cassette that contains all the additional elements required for the expression of the polypeptide in host cells. A typical expression cassette thus contains a promoter operably linked to the nucleic acid sequence encoding the polypeptide and signals required for efficient polyadenylation of the transcript, ribosome binding sites, and translation termination. The nucleic acid sequence encoding the polypeptide is typically linked to a cleavable signal peptide sequence to promote secretion of the polypeptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the recombinant antibody or fusion protein under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

When periplasmic expression of a recombinant protein (e.g., a urodilatin-Fc fragment fusion protein of the present invention) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in, e.g., Gray et al., Gene 39: 247-254 (1985), U.S. Pat. Nos. 6,160,089 and 6,436,674.

As discussed above, a person skilled in the art will recognize that some modifications, especially various conservative substitutions, can be made to an exemplary natriuretic peptide or antibody constant region (e.g., SEQ ID NOs:2 and 4) while still retaining the biological activity of the natriuretic peptide and the desired stabilizing effect of the antibody constant region. Moreover, modifications of a polynucleotide coding sequence may also be made to accommodate preferred codon usage in a particular expression host without altering the resulting amino acid sequence.

### C. Transfection Methods

Standard transfection methods are used to produce bacterial, mammalian, yeast, insect, or plant cell lines that express large quantities of a recombinant polypeptide, which is then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing urodilatin, a C_{H} fragment, or their fusion protein.

### D. Purification of a Recombinantly Produced Polypeptide

Once the expression of a recombinant polypeptide in transfected host cells is confirmed, the host cells are then cultured in an appropriate scale for the purpose of purifying the recombinant polypeptide.

1. Purification of a Recombinant Protein from Prokaryotic and Eukaryotic Cells

When a polypeptide, e.g., a natriuretic peptide-antibody constant region fusion protein of the present invention, is produced recombinantly by transformed bacteria in large amounts, typically after promoter induction, although expression can be constitutive, the polypeptide may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells, e.g., by incubation in a buffer of about 100-150 μg/ml lysozyme and 0.1% Nonidet P40, a non-ionic detergent. The cell suspension can be ground using a Polytron grinder (Brinkman Instruments, Westbury, N.Y.). Alternatively, the cells can be sonicated on ice. Alternate methods of lysing bacteria are described in Ausubel et al. and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2), 1 mM EDTA, 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate, pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides, e.g., a recombinantly produced urodilatin-Fc fragment fusion protein of this invention, from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

2. Standard Protein Separation Techniques for Purification

The following standard protein purification techniques are applicable to both recombinant protein production process in prokaryotic (e.g., bacterial cells) and eukaryotic cells (e.g., mammalian cells). When a recombinant polypeptide, e.g., a natriuretic peptide-antibody constant region fusion protein of the present invention, is expressed in host cells in a soluble form, its purification can follow the standard protein purification procedure described below. This standard purification procedure is also suitable for purifying a natriuretic peptide, an antibody constant region, or their conjugates (including their conjugates joined by a peptide bond, i.e., fusion proteins) obtained from chemical synthesis.

i. Solubility Fractionation

Often as an initial step, and if the protein mixture is complex, an initial salt fractionation can separate many of the unwanted host cell proteins (or proteins derived from the cell culture media) from the recombinant protein of interest, e.g., a urodilatin-Fc fragment fusion protein of the present invention. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the supernatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

ii. Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest, e.g., a natriuretic peptide-antibody constant region fusion protein. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

iii. Column Chromatography

The proteins of interest (such as a natriuretic peptide-antibody constant region fusion protein of the present invention) can also be separated from other proteins on the basis of their size, net surface charge, hydrophobicity, or affinity for ligands. In addition, antibodies raised against the natriuretic peptide or its fusion partner, an antibody constant region, can be conjugated to column matrices and the recombinant polypeptide immunopurified. All of these methods are well known in the art.

It will be apparent to one of skill that chromatographic techniques can be performed at any scale and using equipment from many different manufacturers (e.g., Pharmacia Biotech).

### IV. Chemical Synthesis of a Natriuretic Peptide, an Antibody Constant Region, or Their Fusion Protein

While recombinant production is feasible according the methods described above, a natriuretic peptide and an antibody constant region can also be synthesized chemically using conventional peptide synthesis or other protocols well known in the art, before their conjugation. Furthermore, a natriuretic peptide-antibody constant region fusion protein may also be chemically synthesized as a single polypeptide, especially when the fusion protein is of relatively short length, for instance, less than 150-200 amino acids.

Polypeptides may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art.

Briefly, the C-terminal N-α-protected amino acid is first attached to the solid support. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry, A Practical Textbook, 2nd Ed., Springer-Verlag (1993)).

### V. Chemical Conjugation of a Natriuretic Peptide and Antibody Constant Region

The natriuretic peptide and antibody constant region can be joined by chemical means following their separate production, e.g., recombinant expression and purification, to produce a conjugate of this invention. Chemical conjugation is typically achieved by various covalent bonds including a peptide bond (forming a single fusion protein in some cases) and non-peptide bonds such as a disulfide bond. In the alternative, chemical conjugation can also be achieved by using one or more chemical linkers that connect the natriuretic peptide and the antibody constant region. A large variety of linkers, molecules having multiple functional groups that permit conjugation of two or more compounds via chemical bonds, are known in the art and can be readily obtained from numerous commercial suppliers.

In some cases, chemical modifications can facilitate the conjugation process, including, for example, derivitization for the purpose of linking the natriuretic peptide to the antibody constant region or two antibody constant regions to each other, either directly or through a linking compound, by methods that are well known in the art of protein chemistry. Although covalent bonds are the preferred means of conjugation, in some cases, noncovalent attachment may be used to join a natriuretic peptide and an antibody constant region. The conjugation sites are often at the N- or C-terminus of the peptides, but can also be located in the middle of the peptides via a functional group on an internal amino acid.

The procedure for linking a natriuretic peptide and an antibody constant region will vary according to their amino acid composition and where the peptides are joined. Both peptides, the natriuretic peptide and antibody constant region typically contain a variety of functional groups such as carboxylic acid (—COOH), free amine (—NH₂), or sulfhydryl (—SH) groups, which are available for reaction with a suitable functional group on the other peptide chain to result in a linkage.

Alternatively, a natriuretic peptide or an antibody constant region can be derivatized to expose or to attach additional reactive functional groups. The derivatization may involve attachment of any of a number of linker molecules such as those available from Sigma-Aldrich (St. Louis, Mo.), Pierce Chemical Company (Rockford, Ill.), and Molecular Biosciences (Boulder, Colo.). The linker is capable of forming covalent bonds to both peptide chains of a natriuretic peptide and an antibody constant region. Suitable linkers are well known to those of skill in the art and include, but are not limited to, straight or branched-chain carbon linkers, heterocyclic carbon linkers, or peptide linkers. Since the conjugation partners are polypeptides, the linkers may be joined to the constituent amino acids through their side groups (for example, through a disulfide linkage to cysteine). The linkers may also be joined to the alpha carbon amino and carboxyl groups of the terminal amino acids.

As an alternative means of conjugation, the peptide chains can be joined non-covalently via the interaction of a tag and a tag-binder. The tags and tag-binders can be attached to the natriuretic peptide and antibody constant region by chemical means. For example, synthetic polymers, such as polyurethanes, polyesters, polycarbonates, polyureas, polyamides, polyethyleneimines, polyarylene sulfides, polysiloxanes, polyimides, and polyacetates can form an appropriate tag or tag binder. Suitable pairs for this purpose also include biotin and avidin or streptavidin, and a large number of known cell surface receptor-ligand pairs, e.g., cytokines, cell adhesion molecules, viral proteins, steroids, and various toxins/venoms with their respective receptors. Many of these tags or their coding sequences are commercially available. Other common linkers such as peptides, polyethers, and the like can also serve as tags, and include polypeptide sequences, such as poly-Gly sequences of between about 5 and 200 amino acids. Such flexible linkers are known to persons of skill in the art. For example, poly(ethelyne glycol) linkers are available from Shearwater Polymers, Inc., Huntsville, Ala. These linkers optionally have amide linkages, sulfhydryl linkages, or heterofunctional linkages. Many additional tag/tag binder pairs can also be used for this purpose and would be apparent to one of skill upon review of this disclosure.

One additional alternative is that the two peptide chains can be joined via tag/tag-binder interaction when one of the binding parties is first immobilized to a solid support. Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups that are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:6031-6040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

### VI. Functional Assays for the Conjugates

The natriuretic peptide-antibody constant region conjugates of the present invention are useful for therapeutic applications for conditions known as treatable by the unconjugated natriuretic peptide. Thus, the conjugates retain a portion (e.g., at least 0.1%, 1%, 10%, 20%, 30%, 50% or more) of the biological activity of that natriuretic peptide that is relevant to its medicinal use. Once a conjugate of a natriuretic peptide-antibody constant region is generated, one or more of several functional assays may be performed to assess the level of the desired biological activity retained by the conjugate, the corresponding wild-type natriuretic peptide typically employed in the assay(s) as a control or comparison basis.

### A. NPR Binding Assays

One aspect of a natriuretic peptide's activity is the ability to bind its natural receptor with a high affinity. For instance, urodilatin binds an NPR-A receptor with a high affinity, a urodilatin-antibody constant region conjugate potentially useful according to this invention can therefore be tested in a variety of NPR-A binding assays. One assay format is a cell-free in vitro system, where the NPR-A receptor or its modified version containing its extracellular domain is provided along with a test compound (e.g., a urodilatin conjugate) under conditions permissible for specific binding between the receptor and a wild-type natriuretic peptide such as urodilatin. Another assay format is a cell-based in vitro system, where cells that either naturally express the NPR-A receptor or express the receptor (or a modified version of NPR-A containing its extracellular domain) following transfection are placed in contact with a test compound such as a conjugate of a natriuretic peptide (e.g., urodilatin) and an antibody constant region under conditions permissible for specific binding between the receptor and wild-type natriuretic peptide (e.g., urodilatin). Similar assays can be used for assessing binding affinity for NPR-B and NPR-C. Exemplary assay systems of this type are described in more detail in the scientific literature, e.g., Lowe and Fendly, J. Biol. Chem. 267:21691-21697, 1992, and in a later section of this application.

### B. NPR-A Activation Assays

A further indicator of the activity of a natriuretic peptide is its ability to active its native NPR receptor (e.g., an NPR-A receptor), a guanylyl cyclase, which leads to a detectable increase in intracellular cGMP level. Typically, an assay of this kind is carried out using whole cells that have been transfected to express an NPR receptor (e.g., NPR-A) or a functional variant of the protein. Following exposure of these cells to a test compound (such as a urodilatin-Fc fragment conjugate), their intracellular cGMP concentration is measured and compared against that found in the control cells, which were exposed to a wild-type natriuretic peptide (e.g., urodilatin) or only to a substance known to have no effect on the NPR-A receptor. Similar receptor activation assays can be used for assessing the activation of NPR-B. More detailed description of an NPR activation assay system measuring cGMP level can also be found in Lowe and Fendly, J. Biol. Chem. 267:21691-21697, 1992, and in a later section of this application.

### C. Pharmacokinetic Studies

Pharmacokinetic studies are performed to determine the increased serum half-life of the natriuretic peptide-antibody constant region conjugate of this invention, in comparison with the natriuretic peptide alone. A number of methods are known in the art for measuring in vivo or serum half-life of a test compound, typically carried out in laboratory animals such as rats and mice, by monitoring the serum concentration of the test compound at various time points following the administration of the compound into an animal (e.g., via intravenous injection). For more detailed description of the general methodology, see, e.g., U.S. Pat. Nos. 5,780,054; 6,423,685; and 7,022,673. An increased serum half-life observed in pharmacokinetic experiments utilizing these art-recognized methods is generally accepted as indicative of an increased half-life in human patients.

### VII. Pharmaceutical Compositions and Administration

Natriuretic peptides including urodilatin and others are known for their use in the treatment of various medical conditions such as bacterial infections, pulmonary and bronchial diseases, renal failure, and congestive heart failure, see, e.g., U.S. Pat. Nos. 5,571,789 and 6,831,064, US2005/0089514, and WO2006/110743. Thus, another aspect of the present invention is a pharmaceutical composition comprising a natriuretic peptide-antibody constant region conjugate that retains the therapeutic efficacy of the natriuretic peptide and preferably has a longer serum half-life compared to unconjugated natriuretic peptide alone. This composition, often further containing at least one pharmaceutically acceptable carrier, can be used in therapeutic applications for conditions where the administration of a particular natriuretic peptide is indicated. Pharmaceutical compositions of the invention are suitable for use in a variety of drug delivery systems. Suitable formulations for use in the present invention are found in Remington's Pharmaceutical Sciences, Mack Publishing Company, Philadelphia, Pa., 17th ed. (1985). For a brief review of methods for drug delivery, see, Langer, Science 249:1527-1533 (1990).

For preparing pharmaceutical compositions containing a compound of the present invention, inert and pharmaceutically acceptable excipients or carriers are used. Liquid pharmaceutical compositions include, for example, solutions, suspensions, and emulsions suitable for intradermal, subcutaneous, parenteral, intramuscular, or intravenous administration. Sterile water solutions of the active component (e.g., a urodilatin-Fc fusion protein of this invention) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., a natriuretic peptide-antibody constant region conjugate of this invention) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 5 to 9, more preferably from 7 to 8, and most preferably from 6.5 to 7 or 7.5.

In some embodiments, the compositions can be in solid or semi-solid formulations, using inert ingredients such as gelatin, ascorbate, trehalose, skim milk, starch, xylitol, and the like.

The pharmaceutical compositions of the present invention can be administered by various routes, e.g., subcutaneous, intradermal, transdermal, intramuscular, intravenous, or intraperitoneal. In some cases, the composition is delivered by parenteral, intranasal, topical, oral, or local administration, such as by aerosol or transdermally, for prophylactic treatment. Frequently, the pharmaceutical compositions can be administered locally, e.g., deposited intra-vaginally or intra-rectally. Alternatively, the pharmaceutical compositions can be administered orally. Thus, the invention provides compositions for systemic, local, and oral administration, which comprise a natriuretic peptide-antibody constant region conjugate of this invention dissolved or suspended in a physiologically acceptable carrier, preferably an aqueous carrier, e.g., water, buffered water, saline, PBS, and the like. The compositions may contain pharmaceutically acceptable auxiliary substances as required to approximate physiological conditions, such as pH adjusting and buffering agents, tonicity adjusting agents, wetting agents, detergents and the like.

Alternatively, the composition can be delivered as a suppository or pessary. In some embodiments, the compound of this invention are prepared in a preservation matrix such as described in U.S. Pat. Nos. 6,468,526 and 6,372,209, and are delivered in a dissolvable element made of dissolvable polymer material and/or complex carbohydrate material selected for dissolving properties, such that it remains in substantially solid form before use, and dissolves due to human body temperatures and moisture during use to release the compound in a desired timed release and dosage. See, e.g., U.S. Pat. No. 5,529,782. The compound can also be delivered in a sponge delivery vehicle, such as described in U.S. Pat. No. 4,693,705, or via a tampon-like delivery tube.

In some embodiments, the composition comprising a natriuretic peptide-antibody constant region conjugate (e.g., a urodilatin-Fc fusion protein) of this invention is formulated for oral administration. For example, the physical form of the final recombinant products can be in a tablet/capsule suitable for oral ingestion, optionally in a sustained release formulation.

The preferred route of administering the pharmaceutical compositions is via intravenous or intramuscular injection at weekly dosage of about 0.01-10 g/kg patient body weight, preferably 0.05-5 g/kg, more preferably about 0.1-1 g/kg, of a natriuretic peptide-antibody constant region conjugate for an average adult human patient. The appropriate dose may be delivered in daily, weekly, biweekly, or monthly intervals, by single or multiple administrations of the compositions with dose levels and pattern determined by the treating physician. In any event, the pharmaceutical formulations should provide a quantity of a natriuretic peptide-antibody constant region conjugate of this invention sufficient to effectuate its intended medical use in an individual.

To enhance the therapeutic efficacy of a pharmaceutical composition of this invention, additional ingredients may be included in the composition to provide an additive or synergistic effect. Some examples of such optional ingredients include other therapeutic agents for renal or cardiac conditions already known to those of skill in the art.

### EXAMPLES

The following examples are provided by way of illustration only and not by way of limitation. Those of skill in the art will readily recognize a variety of non-critical parameters that could be changed or modified to yield essentially the same or similar results.

### Methods

1. Generation of Uro-Fc(huFcG1(m1)) and Fc-Uro(huFcG1(m1)) DNA Constructs and Fusion Proteins

The Uro-Fc(huFcG1(m1)) DNA construct was prepared as follows: the signal sequence of proANP (using Genbank sequence NM_{—}006172.1: Homo sapiens natriuretic peptide precursor A (NPPA), mRNA sequence) was generated from genomic DNA using PCR. The primers used for the 3′ end of the signal sequence contained the first 20 bases of the urodilatin sequence and the primer for the 5′ end of urodilatin contained the last 20 bases of the signal sequence. The products of these two PCRs were combined in a second round to amplify the full-length signal sequence plus urodilatin with the addition within the outside primers of a PacI restriction site and partial Kozak sequence (CACC) at the 5′ end and a NotI restriction site at the 5′ end. The resulting product was cleaved with PacI/NotI and ligated into an expression vector containing the huFcG1m(1) sequence using standard molecular biology techniques.

HuFcG1m(1) was produced based on Genbank sequence BC067091 (Homo sapiens immunoglobulin heavy constant gamma 1 (G1m marker), mRNA), and sequence changes were made using standard molecular biology techniques with primers carrying the required changes and the addition of NotI restriction sites at either end. The resulting product was cleaved with NotI and then ligated into an expression vector.

Base changes to BC067091 were made as follows: HuFcG1m(1) starts at position 749 on BC067091. Base changes were at position: 788 C to G, 789 T to C, 791 C to G, 792 T to A, 796 G to C, 798 G to C, 799 A to G, 1154 G to C, 1156 T to G, 1160 C to T, and 1309 C to T.

The Fc-Uro(huFcG1(m1)) DNA construct was prepared as follows: Fc-Uro(huFcG1(m1)) DNA was generated using three pairs of PCR primers and the Uro-Fc(huFcG1(m1)) DNA described above. The 5′ primer of the first pair corresponded to a PacI restriction site followed by the first 20 bases of the signal sequence of proANP (using Genbank sequence NM_{—}006172.1: Homo sapiens natriuretic peptide precursor A (NPPA), mRNA sequence). The 3′ primer of the first pair contained the last 21 bases of the signal sequence plus the first 19 bases of huFcG1m(1) (see above). The 5′ primer of the second primer pair contained the complementary sequence of the first 3′ primer (signal sequence), whereas the 3′ primer of the second pair corresponded to the last 21 bases of huFcG1m(1) plus the first 20 bases of the urodilatin sequence. The 5′ primer of the third set contained the complementary sequence of the 3′ primer of huFcG1m(1), whereas the 3′ primer corresponded to the last 20 bases of the urodilatin sequence followed by a stop codon and a NotI restriction site. The resulting DNA fragment was cleaved with PacI and NotI and subsequently ligated into an expression vector.

Mammalian expression vectors containing the DNA sequences described above were transfected into mammalian tissue culture cells and the proteins encoded by the described DNA sequence purified from the tissue culture medium using standard molecular biology and protein purification techniques.

### 2. Natriuretic Peptide Receptor (NPR) Binding Assays

Kinetics measurements for analytes resulting in estimates of binding affinity constants (K_{D}) were performed using BIAcore 2000 & 3000 instruments and methods recommended by the manufacturer (BIAcore, Sweden). Expression construct encoding for the extracellular domain of the receptor NPR-A fused to the DNA encoding for the Fc domain of human immunoglobulin was expressed in mammalian cells and purified from culture media supernatants using standard molecular biology and protein purification techniques (Bennett et al., J. Biol. Chem. 266(34):23060-23067, 1991). Purified NPR-A-Fc receptor fusion proteins were captured by goat anti-human Fcγ (GAHFc) antibodies (Jackson ImmunoResearch, Cat 109-005-098) immobilized onto the sensorchip surface. The unoccupied GAHFc after the capture was shielded from analytes by the injection of human Fc (huFc, Jackson ImmunoResearch, Cat 009-000-008). Analytes were injected to obtain an association phase followed by injection of HBS-P running buffer (10 mM HEPES, 150 mM sodium chloride, 0.005% P-20 surfactant, pH 7.4) to monitor dissociation for each binding cycle.

The binding kinetics of each analyte-receptor pair was calculated from a global analysis of sensorgram data collected from different analyte concentrations using the BIAevaluate program (BIAcore, Sweden). The affinity (K_{D}) resulting from association (ka) and dissociation (kd) of each analyte against each receptor was obtained by simultaneously fitting the association and dissociation phases of the sensorgram from the analyte concentration series using the same 1:1 Langmuir model from the BIAevaluate software (BIAcore, Sweden).

### 3. Determination of NPR-A Receptor Activation

NPR-A receptor activation by test compounds was determined by incubation with mammalian cells transfected with expression vector DNA encoding for the NPR-A receptor and measuring the amount of cGMP generated in these cells.

Mammalian tissue culture cells (e.g., 3T12 cells) transfected with expression vector DNA encoding for the NPR-A were cultured in 96-well microtiter plates for 18-20 hours (˜50,000 cells/well) using standard tissue culture conditions. The tissue culture medium was then changed to serum-free medium, cells were pre-incubated with 1 mM IBMX for 30 minutes and treated with various concentrations of test compounds for 10 minutes. Cells were then lysed with 0.2 ml/well of 0.1 M HCl for 20 min, and lysates centrifuged at 1000×g for 2 min. The cGMP in the supernatants was determined using a commercial kit (Direct cGMP EIA Kit, Assay Designs, Inc., Cat#901-014) according to the manufacturer's instructions.

### 4. Pharmacokinetic Studies

The in vivo half-life of test compounds was estimated by performing pharmacokinetic studies in rats using standard procedures. All animal procedures were conducted under a protocol approved by the Institutional Animal Care and Use Committee (IACUC) at PDL BioPharma, Inc. PDL BioPharma is accredited by the Association for Accreditation of Laboratory Animal Care International. Groups of male Sprague-Dawley rats with a body weight of 280-355 g were anesthetized with isoflurane and the jugular vein and carotid artery were cannulated for intravenous bolus injections and blood sample collections, respectively. Test compounds were administered as a single intravenous bolus and blood samples drawn at designated time points into tubes containing K₂EDTA and aprotinin. Plasma was prepared from blood samples using standard procedures and stored in aliquots at −80° C. until analysis.

Plasma levels of Uro-Fc (hFcG1(m1)) were determined by sandwich ELISA in 96-well microtiter plates. Briefly, plasma sample dilutions (adjusted with pooled plasma from naïve animals and diluted 1:10 with 10% SuperBlock blocking buffer, Pierce, Rockford. IL) were incubated in a microtiter plate coated with AffiniPure Donkey Anti-Human IgG Fcγ Fragment Specific (Jackson ImmunoResearch Labs, Inc., West Grove, Pa.). Serial dilutions of purified Uro-Fc (hFcG1(m1)) (adjusted with pooled rat plasma and diluted 1:10 with 10% SuperBlock blocking buffer) were used as a standard. After washing the plate, Sheep anti-human Urodilatin Serum (Strategic Biosolutions, Newark, Del.) was added, and after incubation and washing, wells were incubated with Peroxidase-conjugated Donkey anti-Sheep IgG (H+ L) (Jackson ImmunoResearch Labs, Inc., West Grove, Pa.). The ELISA was developed, plates were read in a microtiter plate reader, and concentrations of Uro-Fc (hFcG1(m1)) in plasma samples calculated using standard procedures.

Plasma ularitide concentrations were determined by sandwich ELISA in 96-well microtiter plates. Plates were coated with AffiniPure F(ab′)₂ Fragment Rabbit Anti-Mouse IgG, Fcγ Fragment Specific (Jackson ImmunoResearch Labs, Inc., West Grove, Pa.) followed by washes and incubation with the capturing antibody mouse anti-human Atrial Natriuretic Peptide (ANP) monoclonal antibody abcam 2093 (Abcam, Inc., Cambridge, Mass.). Plasma sample incubations and further steps of the ELISA were performed as described above for the Uro-Fc (hFcG1(m1)) ELISA.

Drug concentration-time data was analyzed with a non-compartmental analysis (NCA) approach using commercialized pharmacokinetic (PK) software WinNonlin 5.2 (Pharsight Corporation; Mountain View, Calif.). Terminal half-life of the drug (t_{1/2}) was calculated from the equation t_{1/2}=ln(2)/λ_{z}, whereas λ_{z} is the terminal elimination rate, estimated from regression of the natural logarithms of the concentrations on the sampling times in the terminal phase.

### Results

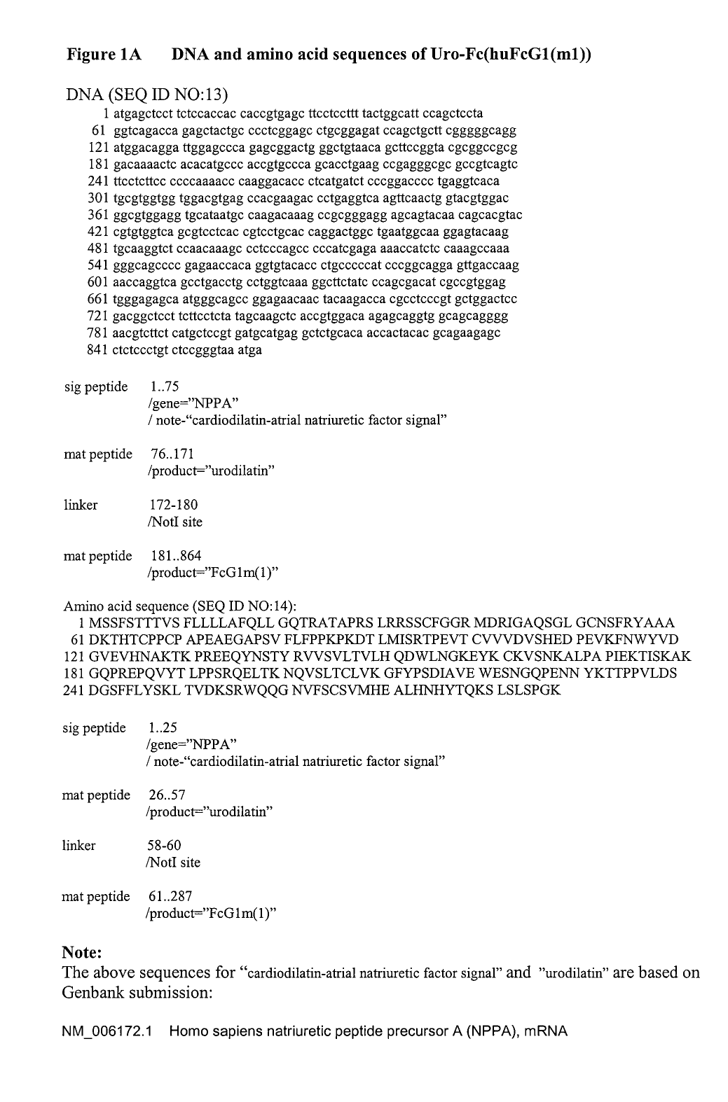

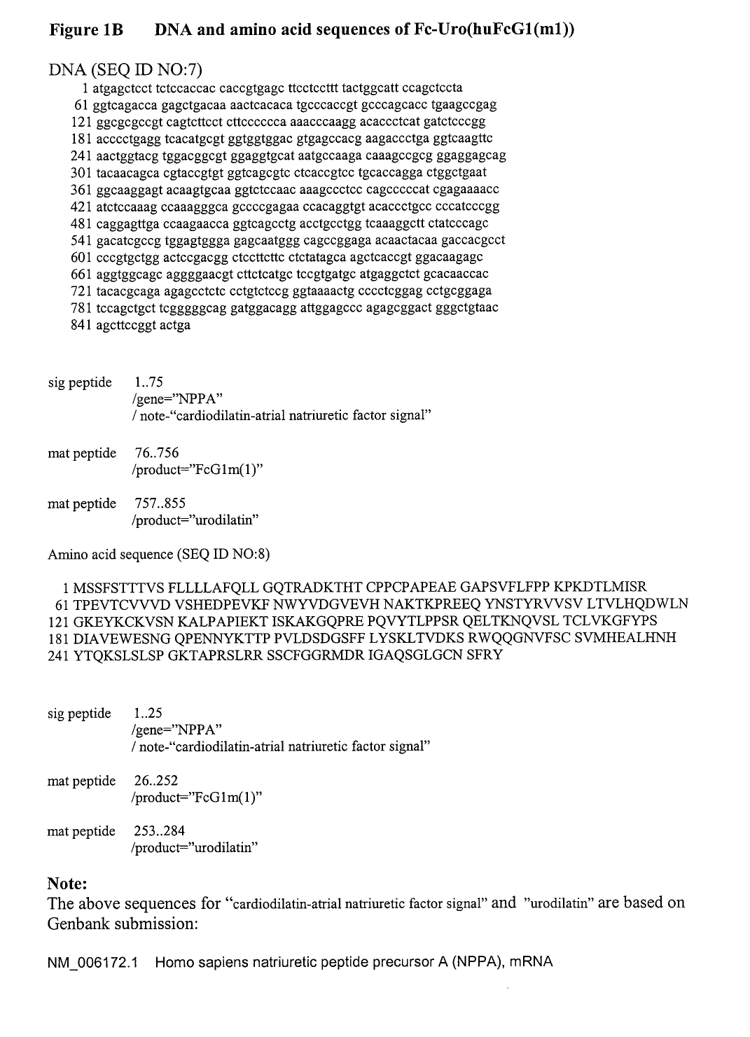

As shown in FIG. 1A, a recombinant DNA fragment encoding for a fusion protein, Uro-Fc(huFcG1(m1)), consisting of the signal sequence of pro-ANP and human urodilatin at the amino terminus, followed by a linker of three amino acid residues, and the Fc portion of a variant of the human immunoglobulin heavy constant gamma 1 region (G1m marker) at the carboxy terminus was generated. FIG. 1B shows recombinant DNA fragment encoding for another fusion protein, Fc-Uro(huFcG1(m1)), consisting of the Fc portion of a variant of the human immunoglobulin heavy constant gamma 1 region (G1m marker) at the amino terminus and human urodilatin at the carboxy terminus. Fusion proteins encoded by these DNA sequences were produced using standard molecular biology and protein purification techniques.

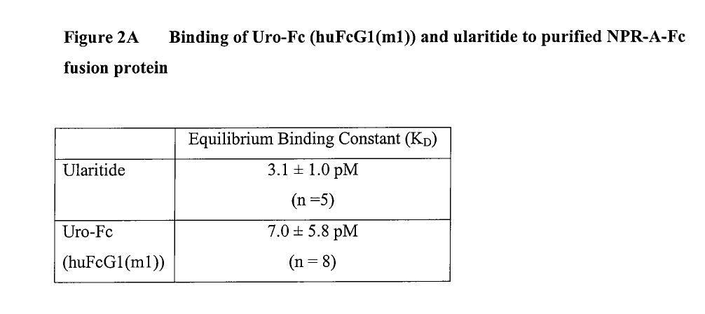

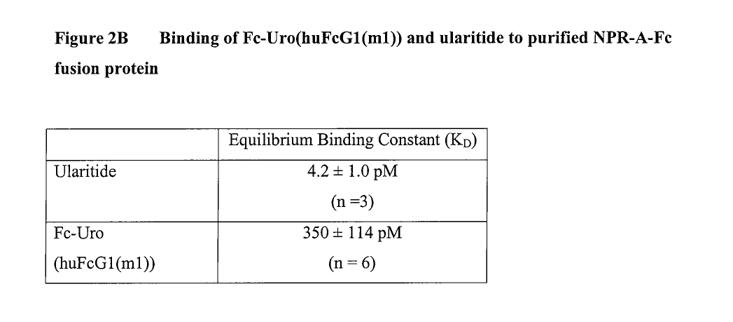

FIG. 2 illustrates the binding of Uro-Fc (huFcG1(m1)) and Fc-Uro(huFcG1(m1)) to purified NPR-A-Fc fusion protein, using ularitide as a control. The receptor binding activity of the purified urodilatin-Fc fusion protein encoded by the Uro-Fc (huFcG1(m1)) DNA was determined in comparison with ularitide using the BIAcore system as described above. The binding measurements were performed with purified Fc fusion protein of the extracellular domain of the receptor NPR-A. Binding affinities (K_{D}) on the NPR-A receptor protein were 3.1±1.0 μM (n=5) for ularitide, and 7.0±5.8 μM (n=8) for the urodilatin-Fc fusion protein. This demonstrates that, similar to ularitide, the Uro-Fc (huFcG1(m1)) fusion protein binds with high affinity to the NPR-A receptor (FIG. 2A). Similarly, receptor binding activity of purified Fc-urodilatin fusion protein encoded by the Fc-Uro(huFcG1(m1)) DNA was also determined in comparison with ularitide using the BIAcore system as described above. Binding affinities (K_{D}) on the NPR-A receptor were 4.2±1.0 pM (n=3) for ularitide, and 0.35±0.1 nM (n=6) for the Fc-urodilatin fusion protein. This demonstrates that the Fc-Uro(huFcG1(m1)) fusion protein binds to the NPR-A receptor (FIG. 2B).

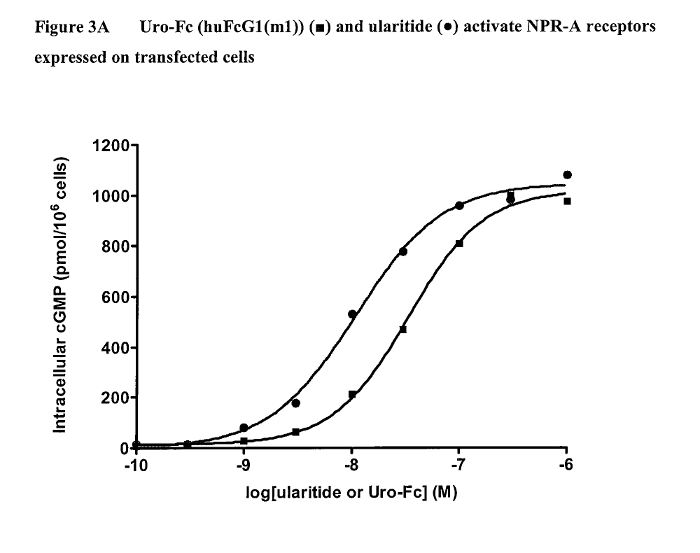

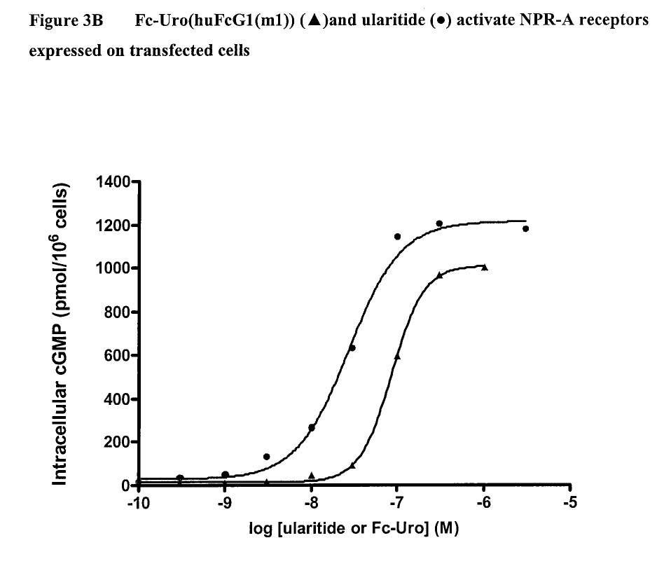

Fusion proteins Uro-Fc (huFcG1(m1)) and Fc-Uro(huFcG1(m1)) were further tested for their ability to activate NPR-A receptors expressed on transfected cells. As shown in FIG. 3, the biological activity of purified urodilatin-Fc fusion proteins encoded by the Uro-Fc (huFcG1(m1)) and Fc-Uro(huFcG1(m1)) DNA were determined in comparison with ularitide. Various concentrations of ularitide and Uro-Fc (huFcG1(m1)) protein were incubated with cells expressing the NPR-A receptor and intracellular cGMP levels were determined according to method described above. Both ularitide and the fusion protein induced cGMP levels in a dose-dependent manner with EC50 of 8.9±2.4 nM and 26.3±7.5 nM, respectively (mean ±SD, n=4 independent experiments). This demonstrates that the Uro-Fc (huFcG1(m1)) fusion protein is biologically active in stimulating the urodilatin receptor NPR-A (FIG. 3A). In a similar fashion, Fc-Uro(huFcG1(m1)) and ularitide were shown activating NPR-A receptors expressed on transfected cells. The biological activity of the purified Fc-urodilatin fusion protein encoded by the Fc-Uro(huFcG1(m1)) DNA was determined in comparison with ularitide according to method described above. Various concentrations of ularitide and Fc-Uro(huFcG1(m1)) protein were incubated with cells expressing the NPR-A receptor and intracellular cGMP levels were determined. Both ularitide and the fusion protein induced cGMP levels in a dose-dependent manner with EC50 of 27 nM and 86 nM, respectively. This demonstrates that the Fc-Uro(huFcG1(m1)) fusion protein is biologically active in stimulating the urodilatin receptor NPR-A (FIG. 3B).

To determine the potential prolongation of the half-life of natriuretic peptide conjugates in vivo, the fusion protein Uro-Fc (hFcG1(m1) and un-conjugated ularitide were subjected to pharmacokinetic studies in rats as described in METHODS. Purified Uro-Fc (hFcG1(m1) was administered as a single intravenous bolus at two doses (5 rats per dose group), 0.41 mg/kg (molar equivalent of 25 ug/kg ularitide), and 2.9 mg/kg (7× molar equivalent of 25 ug/kg ularitide). Blood was drawn from animals at 0.5, 1, 2, 5, 10, 30 minutes, and 1, 3, 6, 24 hours and then every 24 hours until 8 days after the bolus injection. Plasma levels of Uro-Fc (hFcG1(m1) were determined by ELISA and the terminal half-life (t½) calculated. The median t½ was 4.9 hours (range: 1.6-24.7 hours) using a dose of 0.41 mg/kg and 16.1 hours (range: 0.6-31.6 hours) for the dose of 2.9 mg/kg.

In comparison, the half-life of un-conjugated ularitide was determined in rats after single intravenous bolus injection at 25 and 100 ug/kg (4 animals/dose group). Blood was drawn at 0.5, 1, 2, 3.5, 5, 7.5, 10, 20, and 30 minutes after the bolus injection. Terminal half-life was calculated from ularitide plasma levels determined by ELISA. The median t_{1/2} was 0.78 minutes (range: 0.50-0.99 minutes) for 25 ug/kg and 0.83 minutes (range: 0.57-1.16 minutes) for a bolus of 100 ug/kg. These values are very similar to published data generated with a different methodology (0.73 minutes, Abassi, Z. A., et al., 1992, Am. J. Physiol. 263 (Endocrinol. Metab. 26): E870-E876). This demonstrates that the terminal half-life in vivo of the urodilatin fusion protein Uro-Fc (hFcG1(m1) is prolonged compared to un-conjugated ularitide (˜400-fold at the molar equivalent of 25 ug/kg ularitide).

All patents, patent applications, and other publications cited in this application, including published amino acid or polynucleotide sequences, are incorporated by reference in the entirety for all purposes. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

What is claimed is:

1. A conjugate comprising a natriuretic peptide and an antibody constant region, wherein the conjugate binds to a natriuretic peptide receptor.

2. The conjugate of claim 1, wherein the natriuretic peptide is urodilatin.

3. The conjugate of claim 1, wherein the natriuretic peptide receptor is NPR-A.

4. The conjugate of claim 1, wherein the natriuretic peptide and the antibody constant region is linked via a linker.

5. The conjugate of claim 1, which is a fusion polypeptide.

6. The conjugate of claim 5, wherein the fusion polypeptide further comprises a peptide linker.

7. The conjugate of claim 5, wherein the fusion polypeptide comprises the amino acid sequence of SEQ ID NO:6 or 12.

8. The conjugate of claim 1, wherein the antibody constant region is linked to the N-terminus of the natriuretic peptide.

9. The conjugate of claim 1, wherein the antibody constant region is linked to the C-terminus of the natriuretic peptide.

10. The conjugate of claim 1, wherein the natriuretic peptide is linked to an internal amino acid of the antibody constant region.

11. The conjugate of claim 1, which activates a natriuretic peptide receptor.

12. The conjugate of claim 1, which has a longer serum half-life compared with the natriuretic peptide.

13. An isolated nucleic acid comprising a polynucleotide sequence encoding a fusion polypeptide that comprises a natriuretic peptide and an antibody constant region.

14. The nucleic acid of claim 13, wherein the fusion polypeptide further comprises a peptide linker.

15. The nucleic acid of claim 13, wherein the natriuretic peptide is urodilatin.

16. The nucleic acid of claim 13, which comprises the polynucleotide sequence of SEQ ID NO:5 or 11.

17. An expression cassette comprising the nucleic acid of claim 13.

18. An isolated host cell transfected with the expression cassette of claim 17.

19. The host cell of claim 18, which is a eukaryotic cell.

20. A method for recombinantly producing a fusion polypeptide comprising a natriuretic peptide and an antibody constant region, comprising the steps of:
a. transfecting a host cell with the expression cassette of claim 17; and
b. culturing the cell under the condition that are suitable for the cell to express the fusion polypeptide.

21. The method of claim 20, wherein the natriuretic peptide is urodilatin.

22. A composition comprising the conjugate of claim 1 and a pharmaceutically acceptable carrier.

23. A method for treating bacterial infection, pulmonary and bronchial diseases, renal failure, or heart failure, comprising the step of administering to a patient in need thereof an effective amount of the conjugate of claim 1.
